(19) United States
(12) Patent Application Publication
(10) Pub. No.: US 2022/0036978 A1
(43) Pub. Date: Feb. 3, 2022

(54) Systems And Methods For Management Of Clinical Trial Electronic Health Records And Machine Learning Systems Therefor

(71) Applicants: Manoj Pooleery, Ewing, NJ (US); Seth Goodman, Heathrow, FL (US)

(72) Inventors: Manoj Pooleery, Ewing, NJ (US); Seth Goodman, Heathrow, FL (US)

(21) Appl. No.: 17/390,105

(22) Filed: Jul. 30, 2021

Related U.S. Application Data
(60) Provisional application No. 63/059,498, filed on Jul. 31, 2020.

(51) Int. Cl. G16H 10/20; G16H 10/60

(52) U.S. Cl. CPC G16H 10/20; G16H 10/60

(57) ABSTRACT

Systems and methods for managing clinical trial electronic heath records and machine learning systems therefor are provided. An authenticator agent allows healthcare personnel to access and manage clinical trial electronic health records for patients and a patient registry manager enrolls patients in the system. A patient chart exporter electronically communicates with a plurality of electronic health record systems and retrieves patient electronic health records from such systems. A data ingestion, transformation, and analysis engine processes the electronic health records to create a unified clinical trial electronic health record having information about the patient's progress during a clinical trial in a single, easy to access and manage electronic record. Healthcare professionals can electronically annotate the clinical trial electronic health record. A machine learning subsystem processes the clinical trial electronic health records to automatically make recommendations for patients relating to clinical trials.

### RELATED APPLICATIONS

The present application claims the priority of U.S. Provisional Application Ser. No. 63/059,498 filed on Jul. 31, 2020, the entire disclosure of which is expressly incorporated herein by reference.

### BACKGROUND

### Technical Field

The present invention relates generally to electronic medical data systems and methods. More specifically, the present disclosure relates to systems and methods for management of clinical trial electronic health records and machine learning systems therefor.

### Related Art

In today's medical field, electronic records are largely replacing conventional, paper-based medical records. Such systems allow medical professionals to more easily access and manage patient medical information, in addition to reducing storage space requirements attributable to conventional paper-based records. Medical professionals often need only carry a simple computing device such as a laptop, tablet computer, etc., in order to access patient medical data and records when treating a variety of patients during a typical day.

In the clinical trial space, rapid access to, and management of, data and electronic records of patients participating in clinical trials is of paramount importance. One challenge in rapidly and efficiently accessing and managing such records is that they are often stored and maintain in a variety of incompatible data formats, using incompatible electronic health records (“EHR”) programs and systems. As such, a patient's medical data may be stored in a first format utilized by one of the patient's healthcare providers, while the same patient's medical data may be stored in a second format utilized by a second healthcare provider, incompatible with the first provider. Since clinical trials often require careful monitoring of the patient by numerous healthcare providers, and since such healthcare providers often use incompatible EHR systems, it is therefore very difficult to access and manage EHR data from such healthcare providers. Each healthcare provider follows different ways of recording their patients' clinical data. Frequently, the most critical clinical information relating to a patient (which might make the patient eligible for a clinical trial) is specified in a descriptive manner, with various abbreviations of clinical terms. Present EHR systems often lack the ability to conduct rich analytics or perform a search based on the eligibility criteria of a clinical trial on such patient clinical EHR data. They also lack the ability to automatically generate recommendations for patients and/or healthcare providers based on such analytics.

What would be desirable are systems and methods for management of clinical trial electronic health records and machine learning systems therefor, which solve the foregoing and other needs.

### SUMMARY

The present disclosure relates to systems and methods for managing clinical trial electronic heath records and machine learning systems therefor. The system includes an authenticator agent which allows one or more healthcare personnel to access and manage clinical trial electronic health records for one or more patients; a patient registry manager which enrolls one or more patients in the system; a patient chart exporter which electronically communicates with a plurality of electronic health record systems and retrieves patient electronic health records from such systems; and a data ingestion, transformation, and analysis engine which processes the electronic health records to create a unified clinical trial electronic health record having information about the patient's progress during a clinical trial in a single, easy to access and manage electronic record. The system also allows healthcare professionals to electronically annotate the clinical trial electronic health record, and a machine learning subsystem processes the clinical trial electronic health records to automatically make recommendations for patients relating to clinical trials.

### BRIEF DESCRIPTION OF THE DRAWINGS

The foregoing features of the present disclosure will be apparent from the following Detailed Description of the Invention, taken in connection with the accompanying drawings, in which:

FIG. 1 is a diagram illustrating the system of the present invention;

FIG. 2 is flowchart illustrating processing steps carried out by the system for validating users and providing access to the system;

FIG. 3 is flowchart illustrating processing steps carried out by the system for creating patient registries and lists of patients;

FIG. 4 is a flowchart illustrating processing steps carried out by the system for retrieving patient lists, accessing patient electronic health records from disparate electronic health records systems, and creating a consolidated patient record from the disparate electronic health records;

FIG. 5 is a flowchart illustrating processing steps carried out by the system for creating annotated electronic clinical trial records;

FIG. 6 is a flowchart illustrating processing steps carried out by the system for processing electronic clinical trial records using machine learning to automatically generate one or more recommendations relating to a clinical trial; and

FIG. 7 is a diagram illustrating hardware and software components capable of being utilized to implement the systems and methods of the present disclosure.

### DETAILED DESCRIPTION

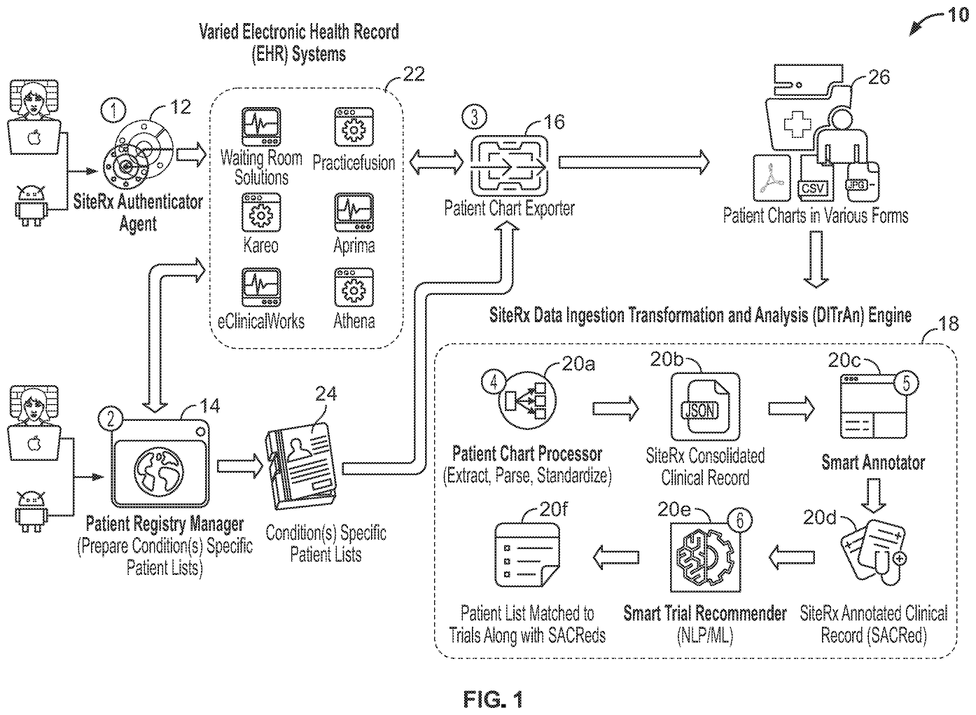

The present disclosure relates to systems and methods for management of clinical trial electronic health records and machine learning systems therefor, as discussed in detail below in connection with FIGS. 1-7.

FIG. 1 is a diagram illustrating the system of the present invention, indicated generally at 10. The system 10 includes various software components including an authenticator agent 12, a patient registry manager 14, a patient chart exporter 16, and a data ingestion, transformation, and analysis engine 18, all of which operate together to provide the processes and features described herein. The authenticator agent 12 authenticates one or more healthcare users of the system, such as doctors, nurses, hospital personnel, healthcare personnel, and potentially non-healthcare users such as insurers, etc. Once such users are authenticated, they can access one or more patient electronic health record (“EHR”) systems 22, which can be maintained using conventional EHR software packages/systems. For example, waiting room personnel at a healthcare facility may utilize an EHR system suitable for tracking patients in waiting rooms, while other EHR systems suitable for different healthcare tasks/actions could also be utilized. Such EHR systems could include, but are not limited to, EHR systems sold under the trademarks ATHENA, PRACTICEFUSION, ECLINICALWORKS, KAREO, APRIMA, as well as other EHR systems. The data storage formats and functions provided by the EHR systems 22 are often incompatible, and as will be discussed below, the system 10 creates a unified clinical EHR for a patient that culls all relevant clinical information from the EHR systems 22 into a single, easy to access and manage clinical trial record that can be annotated by one or more healthcare providers and utilized to automatically generate recommendations relating to clinical trials.

The patient registry manager 14 enrolls one or more patients participating in a clinical trial in the system 10, and automatically prepares condition-specific patient lists 24 for each patient. Such lists 24 are automatically generated by the manager 14 by processing the EHR records 22 to identify the presence of one or more healthcare conditions (e.g., illnesses) indicated in the EHR records 22 which are relevant to one or more clinical trials being conducted (e.g., by a healthcare provider in conjunction with a pharmaceutical company, etc.). The condition-specific patient lists 24 could be stored in any suitable format, such as a database file, a text file, etc.

The patient chart exporter 16 processes both the condition-specific patient lists 24 and data from the EHR systems 22 to identify and retrieve one or more patient charts 26 (patient data records) from one or more of the EHR systems 22. For example, if a particular patient is indicated in the one of the lists 24 as having a cardiovascular condition, the exporter 16 utilizes this information to automatically retrieve charts from various data sources in the EHR systems 22 likely to have information relevant to the patient condition, such as from a hospital EHR system (e.g., if the patient was admitted to a hospital due to a heart attack), a cardiologist's EHR system, and an EHR system operated by the patient's general (internal) medicine practitioner (doctor). As indicated in FIG. 1, the one or more patient charts 26 are stored in various forms/formats that are often incompatible with each other, yet include information about the patient that may be highly relevant to a clinical trial.

The data ingestion and transformation engine 18 receives the patient charts 26, and processes them using a plurality of modules 20a-20e, including a patient chart processor module 20a, a smart consolidated clinical record creation module 20b, a smart annotator module 20c, a clinical record annotation module 20d, a smart trial recommender module 20e, to produce patient lists 20f which are matched to clinical trials alone with relevant consolidated, smart clinical records created by the system 10. The patient chart processor module 20a parses each patient chart 26 (which, as noted above, can be in incompatible forms/formats), extracts relevant information about a particular patient, and formats the extracted data so that it is in a standardized format. The consolidated clinical record creation module 20b receives the standardized data from the module 20a, and creates a consolidated, smart clinical record for each patient. Importantly, the consolidated clinical record includes the relevant information that has been extracted from the incompatible records 26 by the patient chart processor 20a, in an easy to access and manage centralized record for each patient that includes data generated by a plurality of disparate data sources (e.g., doctors, specialists, hospitals, healthcare providers, and other sources). The smart annotator module 20c allows one or more healthcare professionals to make medical (or other) annotations on the consolidated clinical record 20b, creating an annotated clinical record 20d. The smart trial recommender module 20e processes the annotated clinical record 20d using one or more natural language processing (NLP) or machine learning (ML) algorithms to make one or more recommendations relating to one or more clinical trials. For example, the module 20e could process the annotated clinical records 20d to identify patients that may be suitable candidates for a particular clinical trial, based on upon medical, health, or other attributes of the individual that the module 20e learns (via machine learning) from the records 20d. As a result, the module 20e could produce one or more lists 20f that match patients to appropriate clinical trials, including links to such patients' annotated clinical records.

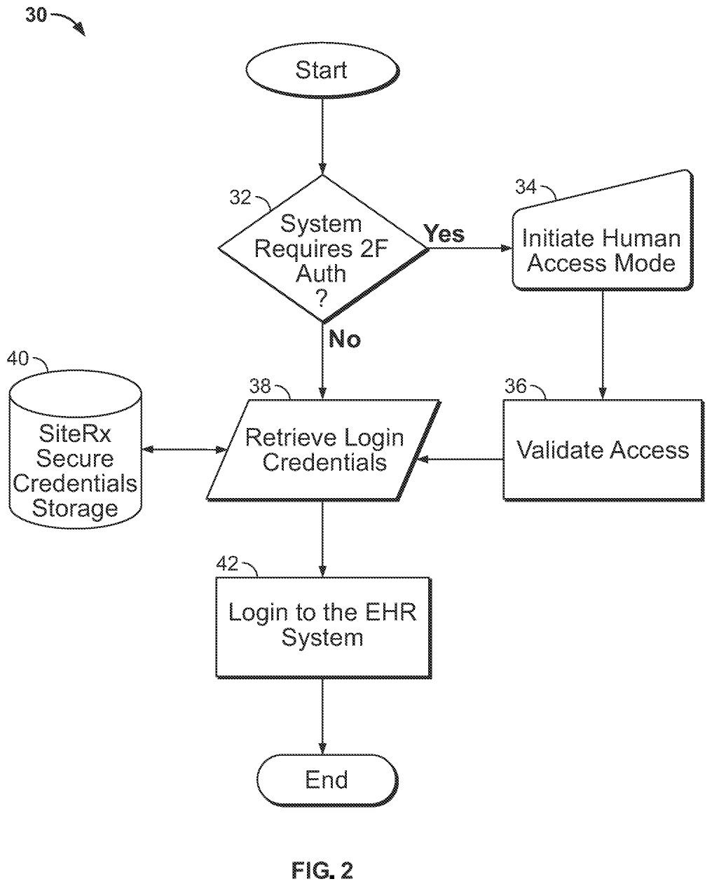

FIG. 2 is flowchart illustrating processing steps carried out by the system for validating users and providing access to the system, indicated generally at 30. Beginning in step 32, the system determines whether the two forms of authentication (“2F”) are required. If so, step 34 occurs, wherein human access mode is initiated (e.g., using biometric identification, etc.). Then, in step 36, the system validates the user based upon the human inputs. If a negative determination is made in step 32, step 38 occurs, wherein the system retrieves the user's login credentials from a secure credentials database 40. Then, in step 42, the user logs into the system (the user's login information is compared to the login credentials to determine whether to grant access to the user).

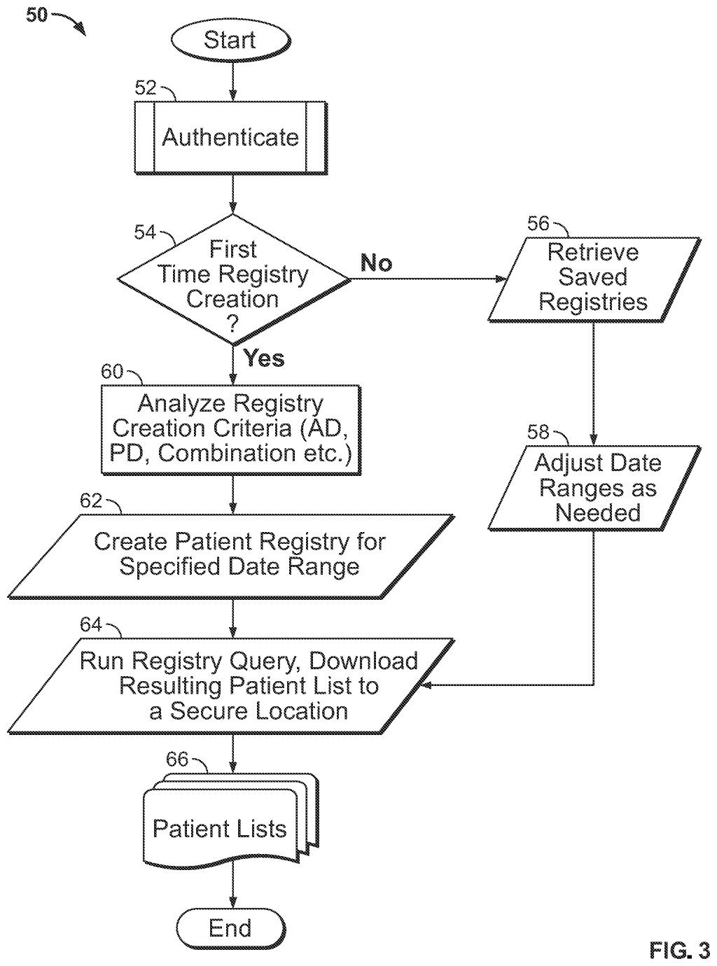

FIG. 3 is flowchart illustrating processing steps carried out by the system for creating patient registries and lists of patients, indicated generally at 50. In step 52, the system authenticates the request for patient registry. In step 54, a determination is made as to whether the present request is the first time a registry has been created. If not, step 56 occurs, wherein the system retrieves a saved registry. Then, in step 58, the system adjusts date ranges as needed, and control is passed to step 64, discussed below. If a positive determination is made in step 54, step 60 occurs, wherein the system analyzes registry creation information specific to one or more chronic conditions such as, but not limited to, Alzheimer's Disease (abbreviated in the drawing as “AD”), Parkinson's Disease (abbreviated in the drawing as “PD”), etc. Then, in step 62, the system creates a patient registry having a specified date range. In step 64, the system runs a registry query that generates lists 66 of registered patients, and downloads the lists 66 to a secure location.

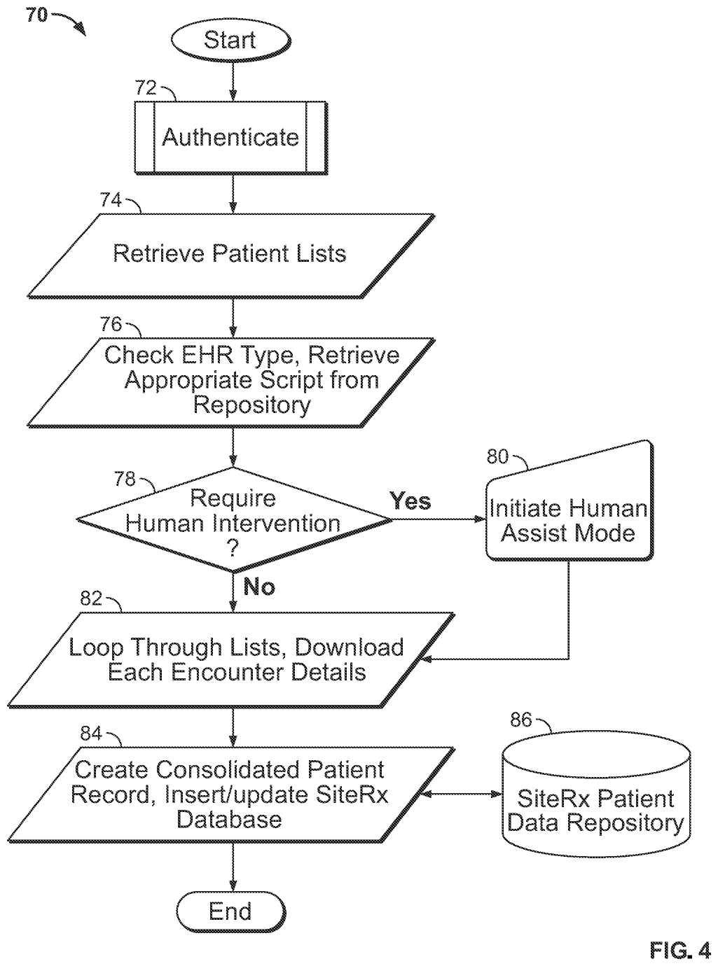

FIG. 4 is a flowchart illustrating processing steps carried out by the system, indicated generally at 70, for retrieving patient lists, accessing patient electronic health records from disparate electronic health records systems, and creating a consolidated patient record from the disparate electronic health records. In step 72, the system authenticates the request for consolidated records. In step 74, the system retrieves patient lists identifying patients for whom records are to be retrieved from the disparate EHR systems 22 of FIG. 1. In step 76, the system processes the lists, checks the EHR types (the types of EHR systems in which the patients' data is stored), and retrieves an appropriate processing script from a repository of scripts. Importantly, each script includes customized software instructions that control how data is retrieved from each EHR system. For example, one script may include customized software instructions for logging into, querying for, and retrieving EHR data from a KAREO EHR system, while another script may include customized software instructions for logging into, querying for, and retrieving EHR data from a PRACTICEFUSION EHR system. Such scripts are rapidly executed and significantly improve the speed with which the system 10 can obtain data from disparate EHR systems.

In step 78, the system determines whether a particular EHR system requires human intervention to facilitate logging into, querying for, and retrieving EHR data from a particular EHR system. If so, step 80 occurs, wherein the system initiates human assistance mode, such that a user of the system can manually log into the EHR system if needed, as well as perform other necessary functions. Such functionality is optional, and most EHR systems can be accessed without human intervention by virtue of the script functionality discussed above. In step 82, the system loops through the retrieved lists to access the various EHR systems that are needed in an automated and rapid fashion, obtaining patient EHR data from such systems and also keeping a log of such activities and successes/failures (referred to in FIG. 4 as “encounter details”). After all applicable EHR systems have been accessed and EHR data obtained therefrom, step 84 occurs, wherein the system creates a consolidated patient record using the EHR data obtained from the disparate EHR systems and stores the consolidated patient record in a data repository 86.

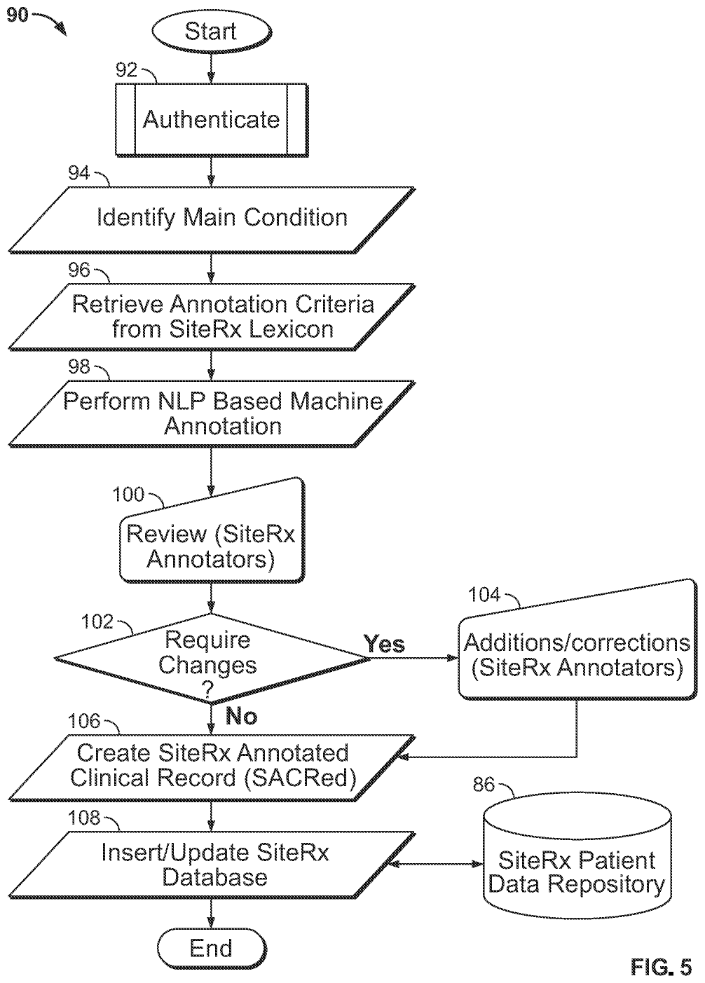

FIG. 5 is a flowchart illustrating processing steps carried out by the system for creating annotated electronic clinical trial records, indicated generally at 90. In step 92, the system authenticates a request to create an annotated clinical trial record. Then, in step 94, the system identifies a main condition of the patient. Such condition could relate to a medical or health condition experienced by the patient, or other condition. In step 96, the system retrieve annotation criteria that are suitable for usage in annotating the patient's consolidated record, based on the condition identified in step 94. In step 98, the system performs NLP-based machine annotation of the record, automatically annotating the record with additional information relating to the patient. In step 100, the system allows a user to review the annotation, and/or to supplement it if desired. In step 102, a determination is made as to whether any changes are required in the annotation. If so, step 104 occurs, wherein the system allows the user to make any required additions or corrections to the annotation. In step 106, the system creates the annotated clinical record which incorporates the annotations automatically made by the system and/or manually by an operator. In step 108, the system inserts/updates the record in a data repository 86.

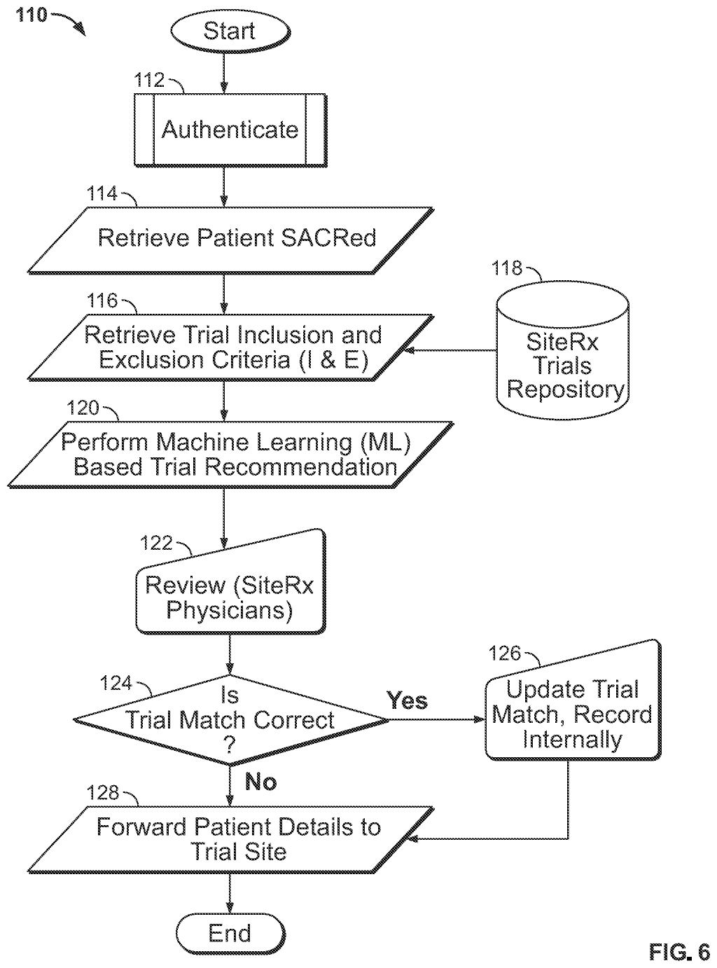

FIG. 6 is a flowchart illustrating processing steps carried out by the system, indicated generally at 110, for processing electronic clinical trial records using machine learning to automatically generate one or more recommendations relating to a clinical trial. In step 112, the system authenticates the request for processing of the clinical trial records. Next, in step 114, the system retrieves an annotated clinical record from the system. Then, in step 116, the system retrieves criteria from a trials database 118 relating to inclusion and exclusion of patients in clinical trials. For example, such criteria could specify particular medical conditions or individual characteristics (e.g., age, weight, etc.) that are required for participation in a clinical trial, or which would militate against participation in a clinical trial. In step 120, the system performs ML processing of the annotated clinical trial record and the criteria to generate a recommendation of whether a patient should participate in a particular clinical trial. In step 122, the recommendation can be reviewed by a healthcare professional, if desired. In step 124, a determination is made as to whether the trial match (recommendation) is correct. If not, step 126 occurs, wherein the trial match is updated as needed. Otherwise, if no correction is required, step 128 occurs, wherein the patient details are forwarded to the trial site (e.g., a website sponsored by the company conducting the clinical trial), so that the clinical trial sponsor can decide whether to invite the recommended patients to participate in the clinical trial.

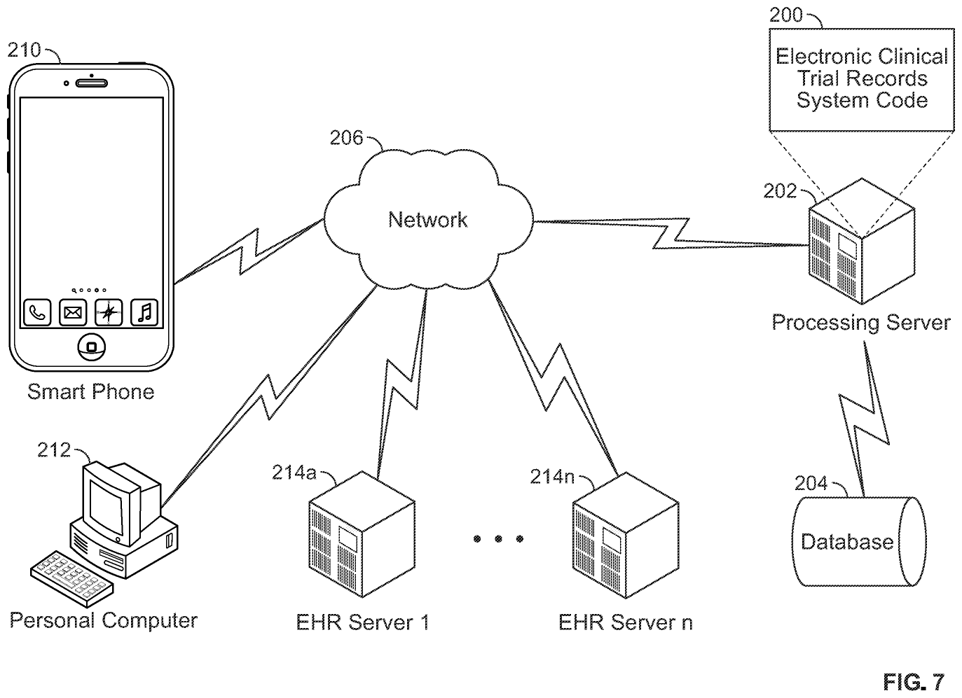

FIG. 7 is a diagram illustrating hardware and software components capable of being utilized to implement the systems and methods of the present disclosure. The processing steps and functions described herein could be embodied as software code executing on a computer system, such as electronic clinical trial records system code 200 that executes on a processing server 202. The code 200 could also communicate with one or more databases 204. The server 202 could be any suitable single-core, multi-core, single-processor, multiple-processor, or other type of computer system, and/or it could be a cloud computing platform, if desired. The server 202 could be accessed over a network 206 using a variety of user computing devices, such as a smart phone 210, a personal computer 212, etc. Additionally, the server 202 can communicate with various disparate EHR systems in the manner described herein, such as EHR servers 214a-214n.

Having thus described the present disclosure in detail, it is to be understood that the foregoing description is not intended to limit the spirit or scope thereof. What is desired to be protected by Letters Patent is set forth in the following claims.

What is claimed is:

1. A system for managing clinical trial electronic health records, comprising:
a memory storing electronic clinical trial records system code; and
a processor in communication with the memory and executing the electronic clinical trial records system code, the processor configured to:
receive a plurality of electronic health records;
process the plurality of electronic health records to extract a plurality of patient charts from the plurality of electronic health records;
process the plurality of patient charts to extract patient data from the plurality of patient charts;
process the patient data to create a plurality of smart clinical records for each patient;
allow a healthcare professional to make an electronic annotation in one or more of the plurality of smart clinical records;
process the plurality of smart clinical records using one or more natural language processing of machine learning algorithms to generate one or more recommendations relating to one or more clinical trials; and
generate and transmit the one or more recommendations relating to the one or more clinical trials.

2. The system of claim 1, wherein the one or more recommendations comprises an identification of one or more candidate patients suitable for a clinical trial.

3. The system of claim 2, wherein the system generates a list of patients for the clinical trial.

4. The system of claim 1, wherein the processor is configured to format the plurality of electronic health records into a standardized format.

5. The system of claim 1, wherein the processor is configured to obtain the electronic health records from a plurality of patient electronic health record systems in communication with the processor.

6. The system of claim 5, wherein the plurality of patient electronic health records are incompatible with each other, and the processor is configured to process the plurality of patient electronic health records into a unified clinical electronic health record.

7. The system of claim 1, wherein the processor is configured to electronically enroll one or more patients in a clinical trial and automatically prepare a condition-specific patient list for each enrolled patient.

8. The system of claim 7, wherein the condition-specific patient list is automatically generated by the processor using the plurality of electronic health records.

9. The system of claim 8, wherein the processor is configured to extract the plurality of patient charts using the plurality of electronic health records and the condition-specific patient list.

10. The system of claim 1, wherein the processor is configured to obtain at least one processing script from a repository of processing scripts and to process at least one of the plurality of electronic health records using the processing script obtained from the repository of processing scripts.

11. The system of claim 10, wherein the at least one processing script instructs the processor how to log into, query for, and retrieve an electronic health record from an electronic health record system in communication with the processor.

12. The system of claim 1, wherein the processor is configured to automatically annotate at least one of the plurality of smart clinical records using natural language processing.

13. A method for managing clinical trial electronic health records, comprising the steps of:
receiving at a processor a plurality of electronic health records;
processing the plurality of electronic health records to extract a plurality of patient charts from the plurality of electronic health records;
processing the plurality of patient charts to extract patient data from the plurality of patient charts;
processing the patient data to create a plurality of smart clinical records for each patient;
allowing a healthcare professional to make an electronic annotation in one or more of the plurality of smart clinical records;
processing the plurality of smart clinical records using one or more natural language processing of machine learning algorithms to generate one or more recommendations relating to one or more clinical trials; and
generating and transmitting the one or more recommendations relating to the one or more clinical trials.

14. The method of claim 13, wherein the one or more recommendations comprises an identification of one or more candidate patients suitable for a clinical trial.

15. The method of claim 14, further comprising generating a list of patients for the clinical trial.

16. The method of claim 13, further comprising formatting the plurality of electronic health records into a standardized format.

17. The method of claim 13, further comprising electronically obtaining the electronic health records from a plurality of patient electronic health record systems in communication with the processor.

18. The method of claim 17, wherein the plurality of patient electronic health records are incompatible with each other, and further comprising the step of processing the plurality of patient electronic health records into a unified clinical electronic health record.

19. The method of claim 13, further comprising electronically enrolling one or more patients in a clinical trial and automatically preparing a condition-specific patient list for each enrolled patient.

20. The method of claim 19, wherein the condition-specific patient list is automatically generated using the plurality of electronic health records.

21. The method of claim 20, further comprising extracting the plurality of patient charts using the plurality of electronic health records and the condition-specific patient list.

22. The method of claim 13, further comprising obtaining at least one processing script from a repository of processing scripts and processing at least one of the plurality of electronic health records using the processing script obtained from the repository of processing scripts.

23. The method of claim 22, wherein the at least one processing script instructs the processor how to log into, query for, and retrieve an electronic health record from an electronic health record system in communication with the processor.

24. The method of claim 13, further comprising automatically annotating at least one of the plurality of smart clinical records using natural language processing.
